(19) United States
(12) Patent Application Publication
(10) Pub. No.: US 2025/0064379 A1
(43) Pub. Date: Feb. 27, 2025

(54) MULTI-BIO-SIGNAL-BASED GERIATRIC COGNITIVE IMPAIRMENT DIAGNOSIS METHOD AND DEVICE

(71) Applicant: SEOUL NATIONAL UNIVERSITY R&DB FOUNDATION, Seoul (KR)

(72) Inventors: Kyoung-Bok MIN, Seoul (KR); Jin-Young MIN, Seoul (KR); Sang-Won HA, Seoul (KR)

(73) Assignee: SEOUL NATIONAL UNIVERSITY R&DB FOUNDATION, Seoul (KR)

(21) Appl. No.: 18/946,398

(22) Filed: Nov. 13, 2024

Related U.S. Application Data
(63) Continuation of application No. PCT/KR2023/007522, filed on Jun. 1, 2023.

(30) Foreign Application Priority Data
Aug. 23, 2022 (KR) 10-2022-0105532

(51) Int. Cl. A61B 5/00; A61B 5/0205; A61B 5/024; A61B 5/11; A61B 5/28; A61B 5/291; A61B 5/372; G16H 50/20

(52) U.S. Cl. CPC A61B 5/4088; A61B 5/02405; A61B 5/112; A61B 5/372; A61B 5/7275; G16H 50/20; A61B 5/0205; A61B 5/28; A61B 5/291; A61B 2503/08; A61B 2562/0219

(57) ABSTRACT

A multi-bio-signal-based geriatric cognitive impairment diagnosis method includes collecting multiple bio-signals including brain waves, heart rate variability, and gait measurement values of a subject during walking; calculating a probability value of a geriatric cognitive impairment disease by using a cognitive impairment diagnosis model, based on the multiple bio-signals; and determining whether there is a geriatric cognitive impairment disease, based on a calculated probability value, wherein the cognitive impairment diagnosis model is a model constructed by applying brain waves, heart rate variability, and gait measurement values of a patient with a geriatric cognitive impairment disease to a logistic function.

### CROSS-REFERENCE TO RELATED APPLICATION

Pursuant to 35 USC 120 and 365(c), this application is a continuation of International Application No. PCT/KR2023/007522 filed on Jun. 1, 2023, and claims the benefit under 35 USC 119(a) of Korean Application No. 10-2022-0105532 filed on Aug. 23, 2022, in the Korean Intellectual Property Office, the entire disclosures of which are incorporated herein by reference for all purposes.

### BACKGROUND

The present disclosure relates to a multi-bio-signal-based geriatric cognitive impairment diagnosis method and device.

Dementia is a representative neurological disease caused by aging. The proportion of the elderly population in Korea is currently much higher than the proportion of the elderly population worldwide, and the incidence of dementia and mild cognitive impairment, which are degenerative geriatric neurological diseases, is expected to increase rapidly in the future. Conventional geriatric cognitive impairment (dementia and mild cognitive impairment) diagnosis technologies have limitations in that the technologies are excessively expensive or invasive due to the use of expensive imaging diagnostic equipment. In addition, questionnaire-based psychological tests have limitations in that the psychological tests are limited to measuring psychological phenomena rather than medical aspects.

Meanwhile, according to a study on spectral analysis of brain waves, an increase in theta activity and a decrease in alpha activity are confirmed in the early stages of Alzheimer's disease. It has been reported that, as Alzheimer's disease progresses, activity increases in the delta and theta frequency bands and activity decreases in the alpha and beta bands. However, it is known that supplementary research is required to be used for established diagnostic purposes.

In addition, studies have illustrated that a decrease in heart rate variability is associated with decreased cognitive function, negative short-term and long-term memory, attention performance, executive function, and executive function, and gait analysis technology is used as an effective tool to study the relationship between motor function and brain function in patients with neurological disorders such as Parkinson's disease, dementia, and stroke.

In relation to this, Korean Patent No. 10-1117770 (Title of the invention: Apparatus for Alzheimer's disease diagnosis using EEG (electroencephalogram) analysis) discloses a device that diagnoses dementia by analyzing the occurrence of brain wave signals in a specific frequency band based on brain wave signals.

In order to overcome the shortcomings of imaging diagnosis or questionnaire diagnosis, the above-described bio-signal-based diagnosis method is being studied, but the existing technology has limitations of using only one signal of the central or peripheral nerves.

Therefore, in order to overcome the limitations of previous studies that diagnose geriatric cognitive impairment by using only a single bio-signal or indicator, such as brain waves, heart rate variability, or gait analysis, a new approach is required to diagnose geriatric cognitive impairment by simultaneously evaluating the entire nervous system, including the central nervous system, autonomic nervous system, and motor function.

### SUMMARY

The present disclosure is intended to solve the above-described problems, and aims to diagnose geriatric cognitive impairment early based on measuring and evaluating multiple bio-signals and indicators that include the entire nervous system including the central nervous system (brain waves), autonomic nervous system (heart rate variability), and motor function (gait and motion analysis).

However, technical tasks to be achieved by the present embodiments are not limited to the technical tasks described above, and there may be other technical tasks.

According to an aspect of the present disclosure, a multi-bio-signal-based geriatric cognitive impairment diagnosis method includes collecting multiple bio-signals including brain waves, heart rate variability, and gait measurement values of a subject during walking; calculating a probability value of a geriatric cognitive impairment disease by using a cognitive impairment diagnosis model, based on the multiple bio-signals; and determining whether there is a geriatric cognitive impairment disease, based on a calculated probability value, wherein the cognitive impairment diagnosis model is a model constructed by applying brain waves, heart rate variability, and gait measurement values of a patient with a geriatric cognitive impairment disease to a logistic function.

According to another aspect of the present disclosure, a multi-bio-signal-based geriatric cognitive impairment diagnosis device includes a data transmission/reception module; a memory storing a geriatric cognitive impairment diagnosis program; and a processor configured to execute the geriatric cognitive impairment diagnosis program stored in the memory, wherein the geriatric cognitive impairment diagnosis program collects multiple bio-signals including brain waves, heart rate variability, and gait measurement values of a subject during walking, calculates a probability value of a geriatric cognitive impairment disease by using a cognitive impairment diagnosis model, based on the multiple bio-signals, and determines whether there is a geriatric cognitive impairment disease, based on a calculated probability value, and the cognitive impairment diagnosis model is a model constructed by applying brain waves, heart rate variability, and gait measurement values of a patient with a geriatric cognitive impairment disease to a logistic function.

According to embodiment of the present disclosure, limitations of cognitive impairment diagnosis using only the known single bio-signal or indicator are overcome, and the accuracy of a geriatric cognitive impairment diagnosis (dementia and mild cognitive impairment) may be increased by multi-analyzing three or more bio-signals, such as the autonomic nerves corresponding to the central and peripheral nerves and motor nerves.

### BRIEF DESCRIPTION OF THE DRAWINGS

Embodiments of the inventive concept will be more clearly understood from the following detailed description taken in conjunction with the accompanying drawings in which:

FIG. 1 is a configuration diagram of a multi-bio-signal-based geriatric cognitive impairment diagnosis device according to an embodiment of the present disclosure;

FIG. 2 is a configuration diagram of a multi-bio-signal-based geriatric cognitive impairment diagnosis device according to an embodiment of the present disclosure;

FIG. 3A, FIG. 3B, and FIG. 3C are views illustrating a method by which each measurement unit measures a bio-signal, according to an embodiment of the present disclosure;

FIG. 4A and FIG. 4B are graphs for comparing distribution results of probability values according to a theoretical logistic function and a geriatric cognitive impairment diagnosis device according to the present disclosure;

FIG. 5 and FIG. 6 illustrate verification results of a geriatric cognitive impairment diagnosis device according to the present disclosure using actual geriatric cognitive impairment confirmation results; and

FIG. 7 is a flowchart illustrating a multi-bio-signal-based geriatric cognitive impairment diagnosis method according to another embodiment of the present disclosure.

### DETAILED DESCRIPTION OF THE EMBODIMENTS

Hereinafter, embodiments of the present disclosure will be described in detail with reference to the attached drawings such that those skilled in the art to which the present disclosure belongs may easily practice the present disclosure. However, the present disclosure may be implemented in various different forms and is not limited to the embodiments described herein. In addition, in order to clearly describe the present disclosure in the drawings, parts that are not related to the description are omitted, and similar components are given similar reference numerals throughout the specification.

In the entire specification of the present disclosure, when a component is described to be “connected” to another component, this includes not only a case where the component is “directly connected” to another component but also a case where the component is “electrically connected” to another component with another element therebetween.

Hereinafter, embodiments of the present disclosure will be described in detail with reference to the attached drawings.

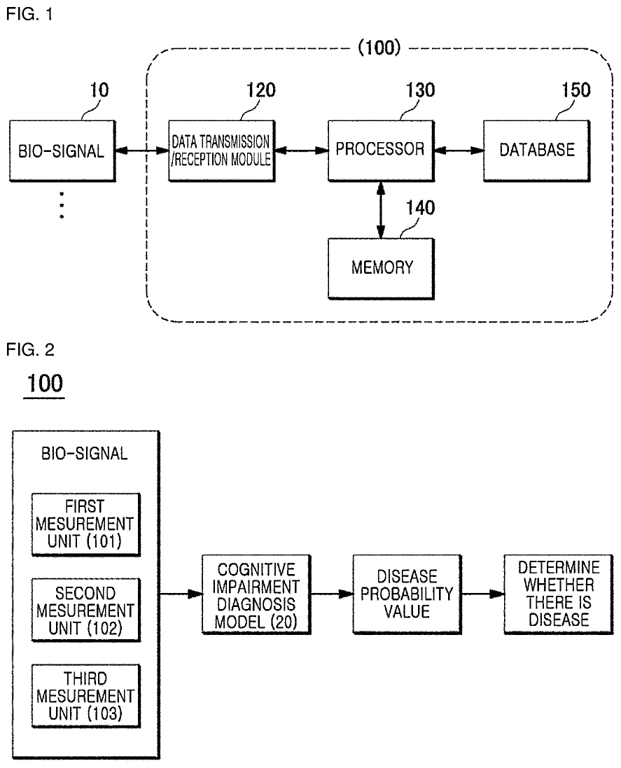

FIG. 1 is a configuration diagram of a multi-bio-signal-based geriatric cognitive impairment diagnosis device according to an embodiment of the present disclosure.

As illustrated in FIG. 1, a geriatric cognitive impairment diagnosis device 100 may include a data transmission/reception module 120, a processor 130, a memory 140, and a database 150.

The data transmission/reception module 120 may receive bio-signals 10 from each measurement unit (not illustrated) and transmit the bio-signals 10 to the processor 130.

The data transmission/reception module 120 may be a device including hardware and software required to transmit and receive signals, such as a control signal and a data signal through wired or wireless connections with other network devices.

The bio-signals 10 transmitted to the data transmission/reception module 120 include brain waves, heart rate variability, and a gait measurement value of a subject. For example, the brain waves refer to an electroencephalography (EEG) signal measured through multiple electrodes that are in contact with the scalp of the subject or are adjacent to the scalp. The heart rate variability refers to heart rate variability (HRV) calculated by analyzing beat-to-beat changes in heart rate based on the ECG signal. The gait measurement values are obtained by measuring a function of motor nerves by using a gait analysis method, and include a stride length and a gait speed represented in numbers and graphs by three-dimensionally analyzing movements of the pelvis, hip joint, knee joint, and ankle joint of the subject in the front, side, and cross sections during a gait cycle.

In this way, each measurement unit may collect brain waves, heart rate variability, and gait measurement values from a subject, and transmit the collected bio-signals 10 to the data transmission/reception module 120. In this way, the collected bio-signals 10 are defined as multiple bio-signals.

The processor 130 executes a cognitive impairment diagnosis program stored in the memory 140, and performs the following processing according to the execution of the cognitive impairment diagnosis program.

The cognitive impairment diagnosis program collects multiple bio-signals including brain waves, heart rate variability, and gait measurement values of a subject, calculates probability of a cognitive disorder disease by using a cognitive disorder diagnosis model based on the multiple bio-signals, and determines whether the subject has a cognitive disorder disease based on the calculated probability value.

Therefore, the present disclosure has an effect of providing a diagnosis of geriatric cognitive disorder (for example, dementia and mild cognitive impairment) with high accuracy by using three or more bio-signals.

The processor 130 may include all kinds of devices capable of processing data. For example, the processor 130 may be a data processing device that is built in hardware and has a physically structured circuit to perform a function represented by a code or command included in cognitive impairment diagnosis the program. For example, the data processing device built in the hardware may include a microprocessor, a central processing unit (CPU), a processor core, a multiprocessor, an application-specific integrated circuit (ASIC), a field programmable gate array (FPGA), or so on, but the scope of the present disclosure is not limited thereto.

The cognitive impairment diagnosis program is stored in the memory 140. The memory 140 stores an operating system for operating the geriatric cognitive impairment diagnosis device 100 or various types of data generated during execution of the cognitive impairment diagnosis program.

In this case, the memory 140 refers to a nonvolatile storage device that maintains the stored information even when power is not supplied and a volatile storage device that requires power to maintain the stored information.

In addition, the memory 140 may perform a function of temporarily or permanently storing the data processed by the processor 130. Here, the memory 140 may include magnetic storage media or flash storage media in addition to the volatile storage device that requires power to maintain the stored information, but the scope of the present disclosure is not limited thereto.

The database 150 stores or provides data required for the geriatric cognitive impairment diagnosis device 100 under the control of the processor 130. For example, the database 150 may store the probability of a cognitive impairment disease detected by using a cognitive impairment diagnosis model based on multiple bio-signals. This database 150 may be included as a separate component from the memory 140, or may be constructed in a part of the memory 140.

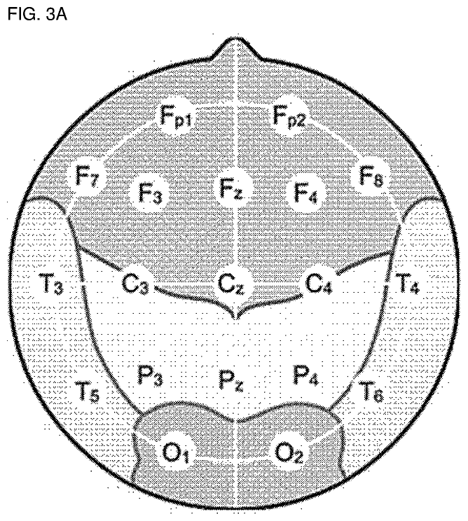

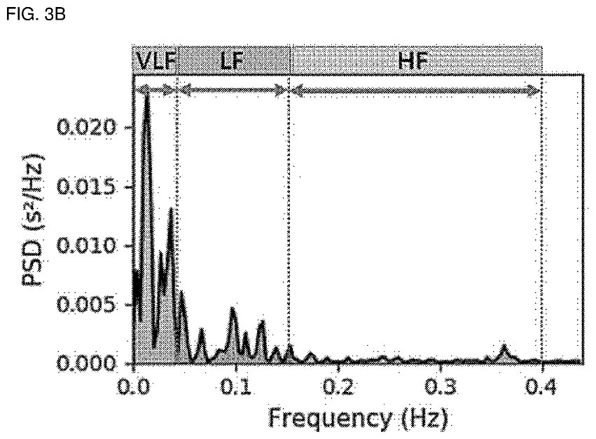

FIG. 2 is a structural diagram of multi-bio-signal-based geriatric cognitive impairment diagnosis device according to an embodiment of the present disclosure. FIGS. 3A to 3C are views illustrating a method of measuring bio-signals by using each measurement unit, according to an embodiment of the present disclosure.

Specifically, referring to FIG. 2, the geriatric cognitive impairment diagnosis device 100 executes a program that collects multiple bio-signals including brain waves, heart rate variability, and gait measurement values of a subject, calculates a cognitive impairment disease probability value by using a cognitive impairment diagnosis model 20 based on the multiple bio-signals, and determines whether the subject has a cognitive impairment disease, based on the calculated probability value.

For example, referring to FIG. 3A, the program calculates a first measurement value by analyzing a frequency spectrum based on the brain waves measured through multiple electrodes in contact with the scalp of the subject, calculates a second measurement value by analyzing a frequency spectrum based on the heart rate variability measured through multiple electrodes in contact with the skin of the subject, and calculates a third measurement value by analyzing the gait measurement values including a stride length and a gait speed measured through multiple motion detection sensors during a gait cycle of the subject

Specifically, a first measurement unit 101 may measure the brain waves through 17 electrodes in six regions corresponding to the frontal lobe, temporal lobe, occipital lobe, parietal lobe, prefrontal lobe, and central gyrus of the subject. In this case, the first measurement unit 101 measures spectral power according to a quantitative brain wave analysis technique as the first measurement value. Here, a spectrum is divided into frequency bands of delta (1-4 Hz), theta (4-8 Hz), alpha (8-12 Hz), beta (12-25 Hz), high beta (25-30 Hz), and gamma (30-40 Hz) depending on clinical classification of the brain waves. Accordingly, the first measurement value includes the spectral power for each frequency band. In addition, the first measurement value further includes a ratio between respective spectral powers, and may include a delta-alpha ratio, a theta-alpha ratio, and a theta-beta ratio. In addition, the first measurement value may include coherence which means a relationship with a measurement site for each frequency. For example, the first measurement value may include spectral powers for each frequency band, such as a delta wave spectrum (occipital lobe): 0.442191, a theta wave spectrum (occipital lobe): 0.002762, an alpha wave spectrum (occipital lobe): 0.001108, a beta wave spectrum (occipital lobe): 0.000003, a high beta wave spectrum (occipital lobe): 0.000437, a gamma wave spectrum (occipital lobe): 0.000463, and may include ratios between respective spectral powers, such as a delta-alpha ratio: 1.000, a theta-alpha ratio: 0.993, and a theta-beta ratio: 0.997.

Referring to FIG. 3B, the second measurement unit 102 numerically measures an HRV result obtained by analyzing a beat-to-beat variation of a heart rate based on the ECG signal of a subject. Here, the HRV means a periodic change of the heart rate over time and reflects an overall activity of an autonomic nervous system because the HRV is adjusted by the sympathetic nerve and parasympathetic nerve. That is, the second measurement unit 102 analyzes the HRV as the second measurement value and measures a time variation between heartbeats and the spectral power. Accordingly, the second measurement value is divided into indices of time domain analysis, frequency domain analysis, and nonlinear analysis. For example, the time domain analysis includes an average heart rate, a maximum heart rate, a minimum heart rate, standard deviation of all NN intervals (SDNN), percent of NN interval over 50 ms), and so on, and the frequency domain analysis includes a very low frequency (VLF, 0.003-0.04 Hz), a low frequency (LF, 0.04-0.15 Hz), a high frequency (HF, 0.15-0.4 Hz), and so on. In addition, the nonlinear analysis includes approximate entropy (APEN), sample entropy (SAEN), and so on. For example, the second measurement value may include a frequency domain analysis index, such as low frequency: 109.4, a high frequency: 70.4, and an ultra-low frequency: 1335.6, and a time domain analysis index, such as an average heart rate: 68.6, a minimum heart rate: 62.4, a maximum heart rate: 74.1, SDNN: 3.8, and pnn50: 0.003.

Referring to FIG. 3C, the third measurement unit 103 measures numerical values and graphs, as the third measurement value, obtained by three-dimensionally analyzing movements of the pelvis, hip joint, knee joint, and ankle joint of a subject in the front, side, and cross sections during a gait cycle. Accordingly, the third measurement value includes a stride length and a gait speed, is obtained by quantitatively measuring each element, and includes quantitative proportional values of the stride length and gait speed. For example, the third measurement value may include the quantitative proportional values of the stride length and gait speed, such as stride time: 515.7, long stride time: 1012.9, steps per minute: 118.5, stride: 169.4, and gait speed: 6.1.

The cognitive impairment diagnosis model 20 is a model constructed by applying brain waves, HRV, and gait measurement values of a patient with senile cognitive impairment to a logistic function, and is constructed through a process of determining an estimator that constitutes the logistic function based on actual data on a patient with senile cognitive impairment. The cognitive impairment diagnosis model 20 includes a logistic function for calculating a probability value of geriatric cognitive impairment by using variables including the first measurement value, the second measurement value, and the third measurement value.

In this case, the logistic function may further include a fourth measurement value as a variable. For example, the fourth measurement value may include age, gender, and education level as demographic variables. For example, the fourth measurement value may include demographic variables, such as late elderly: 75 years or older (1), gender: female (0), and education level: high school graduate or lower (0).

For example, the cognitive impairment diagnosis model 20 may calculate statistics according to Equation 1 and estimate a correlation between the collected multiple bio-signals (first to third measurement values) and geriatric cognitive impairment according to the logistic function defined by Equation 2. That is, the estimated correlation is generated as an estimation value β for each variable. Next, an actual measured measurement value x and an estimation value β for each variable are multiplied and all values are added up.

$$Y = {\sum }_{i = 1}^{n} {β}_{i} {X}_{{EEG}_{i}} + {\sum }_{j = 1}^{n} {β}_{i} {X}_{{HRV}_{i}} + {\sum }_{k = 1}^{n} {β}_{i} {X}_{{GAIT}_{i}} + {\sum }_{l = 1}^{n} {β}_{i} {X}_{{DEMO}_{i}} \qquad Equation 1$$

where, XEEG is the first measurement value, XHRV is the second measurement value, XGAIT is the third measurement value, XDEMO is the fourth measurement value, EEG is brain waves, HRV is heart rate variability, GAIT is gait analysis, DEMO is a demographic variable, and β means the estimation value for each variable.

$$ln ( \frac{P}{1 - P} ) = {\sum }_{i = 1}^{n} {β}_{i} {X}_{{EEG}_{i}} + {\sum }_{j = 1}^{n} {β}_{i} {X}_{{HRV}_{i}} + {\sum }_{k = 1}^{n} {β}_{i} {X}_{{GAIT}_{i}} + {\sum }_{l = 1}^{n} {β}_{i} {X}_{{DEMO}_{i}} \qquad Equation 2$$

where, P is a probability value that a subject (patient) has geriatric cognitive impairment (disease) and has a value between 0 and 1.

Next, the cognitive impairment diagnosis model 20 may calculate the probability value P according to Equation 3 and Equation 4. First, a value summed by Equation 2 is exponentially transformed according to Equation 3. Thereafter, the disease probability value P is further transformed according to Equation 4.

$$( \frac{P}{1 - P} ) = {e}^{{\sum }_{i = 1}^{n} {β}_{i} {X}_{{EEG}_{i}} + {\sum }_{j = 1}^{n} {β}_{i} {X}_{{HRV}_{i}} + {\sum }_{k = 1}^{n} {β}_{i} {X}_{{GAIT}_{i}} + {\sum }_{l = 1}^{n} {β}_{i} {X}_{{DEMO}_{i}}} \qquad Equation 3$$

$$P = \frac{{e}^{{\sum }_{i = 1}^{n} {β}_{i} {X}_{{EEG}_{i}} + {\sum }_{j = 1}^{n} {β}_{i} {X}_{{HRV}_{i}} + {\sum }_{k = 1}^{n} {β}_{i} {X}_{{GAIT}_{i}} + {\sum }_{l = 1}^{n} {β}_{i} {X}_{{DEMO}_{i}}}}{1 - {e}^{{\sum }_{i = 1}^{n} {β}_{i} {X}_{{EEG}_{i}} + {\sum }_{j = 1}^{n} {β}_{i} {X}_{{HRV}_{i}} + {\sum }_{k = 1}^{n} {β}_{i} {X}_{{GAIT}_{i}} + {\sum }_{l = 1}^{n} {β}_{i} {X}_{{DEMO}_{i}}}} \qquad Equation 4$$

That is, the transformed result value is determined as the final disease probability value P by Equation 3 and Equation 4.

Thereafter, the program determines whether there is a cognitive disorder disease based on the disease probability value P calculated through the cognitive disorder diagnosis model 20. For example, as defined by Equation 5, when the result value is less than 1 or more than 0.5, it can be diagnosed as a cognitive disorder disease, and when the result value is greater than or equal to 0 and less than or equal to 0.5, it can be diagnosed as normality.

$$decision ( x ) = \{ \begin{matrix}1 & if P ( y = 1 \vert x ) > 0.5 \\ 0 & otherwise\end{matrix} \qquad Equation 5$$

In other words, as illustrated in FIG. 2, the geriatric cognitive disorder diagnosis device 100 inputs multiple bio-signals collected for a subject to the cognitive disorder diagnosis model 20, and the cognitive disorder diagnosis model 20 calculates the disease probability value P based on the measured values and estimated values for each independent variable (brain waves, HRV, gait analysis (a stride length and gait speed), and demographic variables) by Equation 1 to Equation 4 described above. In addition, by Equation 5, it is possible to determine whether a subject has geriatric cognitive impairment based on the disease probability value P.

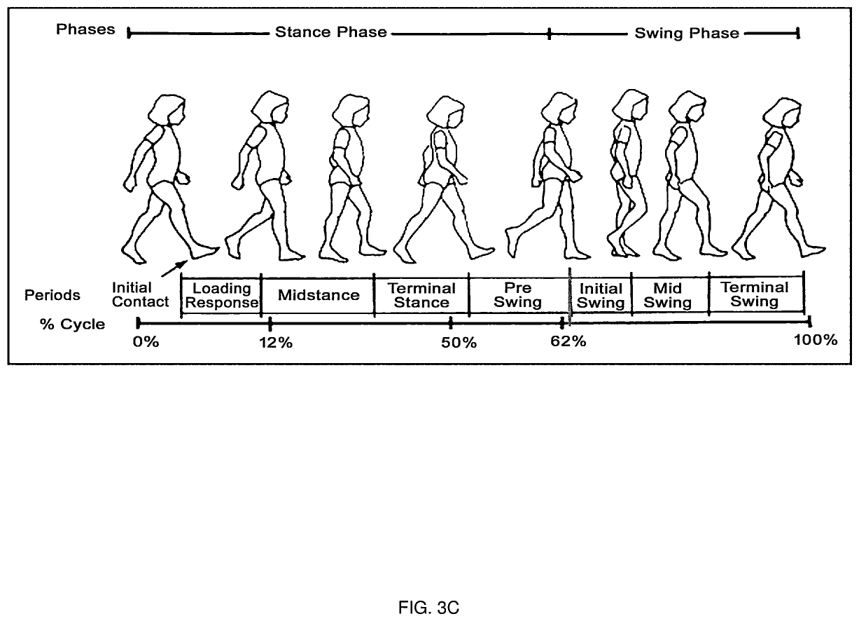

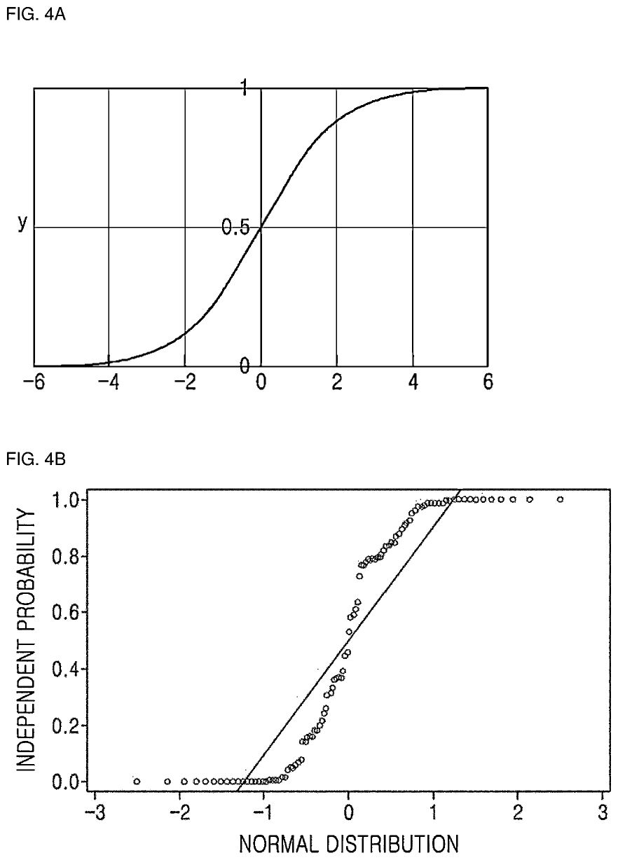

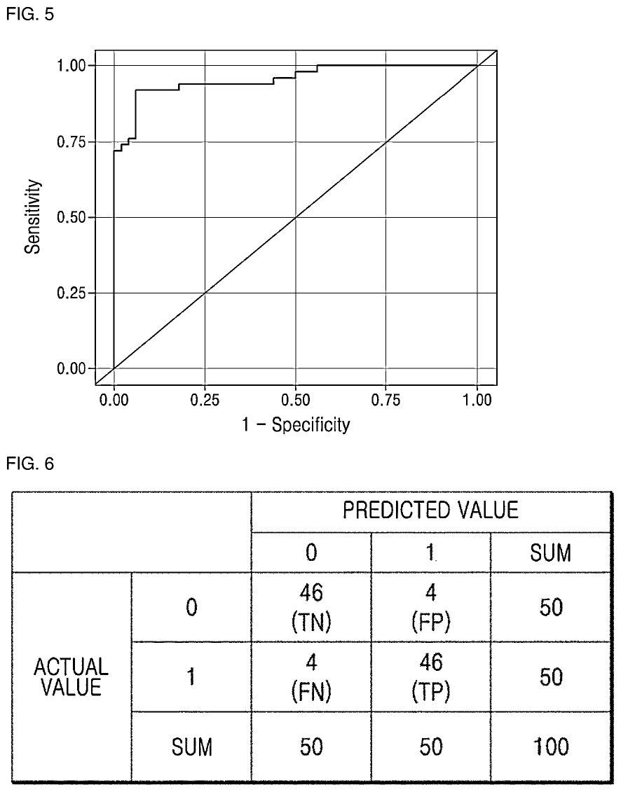

FIG. 4A and FIG. 4B are graphs for comparing distribution results of probability values according to a theoretical logistic function and a geriatric cognitive impairment diagnosis device according to the present disclosure, and FIG. 5 and FIG. 6 illustrate verification results of a geriatric cognitive impairment diagnosis device according to the present disclosure using actual geriatric cognitive impairment confirmation results.

FIG. 4A is a graph illustrating a distribution of probability values of a theoretical logistic function (logit function), and FIG. 4B is a graph illustrating a distribution result of probability values of geriatric cognitive impairment by using the geriatric cognitive impairment diagnosis device 100 of the present disclosure which is obtained by targeting 100 elderly people aged 65 or older.

As illustrated in FIG. 4A and FIG. 4B, distribution results of the probability values of the geriatric cognitive impairment diagnosis device 100 based on the measured values and estimated values for 100 subjects show a similar pattern to a theoretical distribution of a logistic function.

FIG. 5 illustrates a receiver-operating characteristic (ROC) curve of geriatric cognitive impairment diagnosis, as a result of applying the geriatric cognitive impairment diagnosis device 100 by targeting 100 elderly people aged 65 or older. In this case, the ROC curve is 0.955, indicating excellent prediction performance.

FIG. 6 is a table illustrating accuracy of the geriatric cognitive impairment diagnosis device 100 of the present disclosure by comparing a geriatric cognitive impairment diagnosis (an actual value) diagnosed by a clinical apparatus using a confusion matrix and a geriatric cognitive impairment diagnosis (a predicted value) according to the present disclosure.

In this case, the accuracy is calculated as Accuracy =(TP+TN)/(TP+FP+TN +FN)=(46+46)/(46+4+46+4)=0.92, and the accuracy of the calculated disease diagnosis is 92%.

As such, it can be seen that a disease diagnosis result of the geriatric cognitive impairment diagnosis device 100, to which a logistic function of the present disclosure is applied, is highly reliable.

Hereinafter, description of the same configuration among the configurations illustrated in FIGS. 1 to 6 described above is omitted.

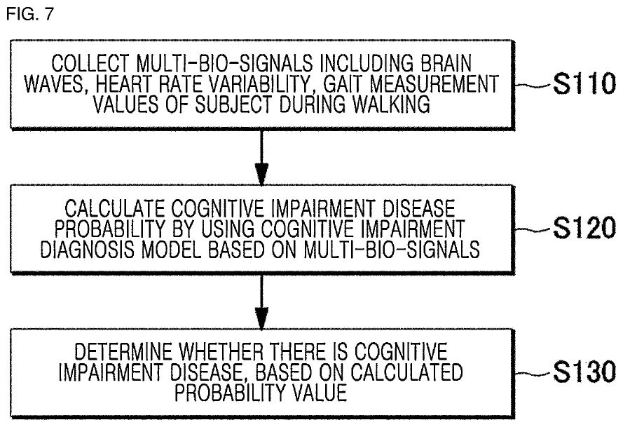

FIG. 7 is a flowchart illustrating a multi-bio-signal-based geriatric cognitive impairment diagnosis method according to another embodiment of the present disclosure.

Referring to FIG. 7, the multi-bio-signal-based geriatric cognitive impairment diagnosis method using the multi-bio-signal-based geriatric cognitive impairment diagnosis method and device 100, according to another embodiment of the present disclosure, includes step S110 of collecting multi-bio-signals including brain waves, HRV, and gait measurement values of a subject, step S120 of calculating a cognitive impairment disease probability by using the cognitive impairment diagnosis model 20 based on the multi-bio-signals, and step S130 of determining whether there is a cognitive impairment disease, based on a calculated probability value. In this case, the cognitive impairment diagnosis model 20 is a model constructed by applying brain waves, HRV, and gait measurement values of a patient with geriatric cognitive impairment disease to a logistic function.

Step S110 includes a step of calculating a first measurement value by analyzing a frequency spectrum based on the brain waves measured through multiple electrodes in contact with the scalp of a subject, a step of calculating a second measurement value by analyzing a frequency spectrum based on HRV measured through multiple electrodes in contact with the skin of the subject, and a step of calculating a third measurement value by analyzing gait measurement values including a stride length and a gait speed measured through multiple motion detection sensors during a gait cycle of the subject.

The cognitive impairment diagnosis model 20 includes a logistic function for calculating a probability value of a geriatric cognitive impairment disease by using variables including the first measurement value, the second measurement value, and the third measurement value.

In step S120, the cognitive impairment diagnosis model 20 may estimate a correlation between the multiple bio-signals measured by each measurement unit and geriatric cognitive impairment according to the logistic function previously defined as in Equation 1 to Equation 4 described above, and generate an estimated amount β to be assigned to each collected measurement value. In addition, for each variable (a brain wave, HRV, and a gait measurement value), an actual measured value x may be multiplied by the estimated value β, and then all values may be summed. Then, the summed value may be transformed to an exponential value and undergoes an additional conversion process to calculate a disease probability value P.

Next, in step S130, whether there is a cognitive disorder disease is determined based on the calculated disease probability value P, and when the disease probability value P is less than 1 and greater than 0.5, it can be diagnosed as a cognitive disorder disease according to the defined Equation 5, and when the disease probability value P is greater than or equal to 0 and less than or equal to 0.5, it can be diagnosed as normality.

Embodiments of the present disclosure may be performed in the form of a recording medium including instructions executable by a computer, such as a program module executed by a computer. A computer readable medium may be any available medium that may be accessed by a computer and includes both volatile and nonvolatile media, removable and non-removable media. Also, the computer readable medium may include a computer storage medium. A computer storage medium includes both volatile and nonvolatile media and removable and non-removable media implemented by any method or technology for storing information, such as computer readable instructions, data structures, program modules or other data.

In addition, although a method and device of the present disclosure are described with respect to specific embodiments, some or all of components or operations thereof may be implemented by using a computer system having a general-purpose hardware architecture.

The above description of the present disclosure is for illustrative purposes only, and those skilled in the art to which the present disclosure belongs will understand that the present disclosure may be easily modified into another specific form based on the descriptions given above without changing the technical idea or essential features of the present disclosure. Therefore, the embodiments described above should be understood as illustrative in all respects and not limiting. For example, each component described as single may be implemented in a distributed manner, and similarly, components described as distributed may also be implemented in a combined form.

The scope of the present disclosure is indicated by the claims described below rather than the detailed description above, and all changes or modified forms derived from the meaning and scope of the claims and their equivalent concepts should be construed as being included in the scope of the present disclosure.

What is claimed is:

1. A multi-bio-signal-based geriatric cognitive impairment diagnosis method which is performed by a computer, the multi-bio-signal-based geriatric cognitive impairment diagnosis method comprising:
collecting multiple bio-signals including brain waves, heart rate variability, and gait measurement values of a subject;
calculating a probability value of a geriatric cognitive impairment disease by using a cognitive impairment diagnosis model, based on the multiple bio-signals; and
determining whether there is a geriatric cognitive impairment disease, based on a calculated probability value,
wherein the cognitive impairment diagnosis model is a model constructed by applying brain waves, heart rate variability, and gait measurement values of a patient with a geriatric cognitive impairment disease to a logistic function.

2. The multi-bio-signal-based geriatric cognitive impairment diagnosis method of claim 1, wherein the collecting of the multiple bio-signals includes:
calculating a first measurement value by analyzing a frequency spectrum based on brain waves measured through a plurality of electrodes in contact with a scalp of the subject;
calculating a second measurement value by analyzing a frequency spectrum based on heart rate variability measured through a plurality of electrodes in contact with a skin of the subject; and
calculating a third measurement value by analyzing gait measurement values including a stride length and a gait speed measured through a plurality of motion detection sensors during a gait cycle of the subject.

3. The multi-bio-signal-based geriatric cognitive impairment diagnosis method of claim 2, wherein
the cognitive impairment diagnosis model includes a logistic function for calculating the probability value of the geriatric cognitive impairment disease by using variables including the first measurement value, the second measurement value, and the third measurement value.

4. A multi-bio-signal-based geriatric cognitive impairment diagnosis device comprising:
a data transmission/reception module;
a memory storing a geriatric cognitive impairment diagnosis program; and
a processor configured to execute the geriatric cognitive impairment diagnosis program stored in the memory,
wherein the geriatric cognitive impairment diagnosis program collects multiple bio-signals including brain waves, heart rate variability, and gait measurement values of a subject, calculates a probability value of a geriatric cognitive impairment disease by using a cognitive impairment diagnosis model, based on the multiple bio-signals, and determines whether there is a geriatric cognitive impairment disease, based on a calculated probability value, and
the cognitive impairment diagnosis model is a model constructed by applying brain waves, heart rate variability, and gait measurement values of a patient with a geriatric cognitive impairment disease to a logistic function.

5. The multi-bio-signal-based geriatric cognitive impairment diagnosis device of claim 4, wherein
the geriatric cognitive impairment diagnosis program calculates a first measurement value by analyzing a frequency spectrum based on brain waves measured through a plurality of electrodes in contact with a scalp of the subject, calculates a second measurement value by analyzing a frequency spectrum based on heart rate variability measured through a plurality of electrodes in contact with a skin of the subject, and calculates a third measurement value by analyzing gait measurement values including a stride length and a gait speed measured through a plurality of motion detection sensors during a gait cycle of the subject.

6. The multi-bio-signal-based geriatric cognitive impairment diagnosis device of claim 5, wherein
the cognitive impairment diagnosis model includes a logistic function for calculating the probability value of the geriatric cognitive impairment disease by using variables including the first measurement value, the second measurement value, and the third measurement value.

7. A non-transitory computer-readable recording medium on which a computer program for performing a multi-bio-signal-based geriatric cognitive impairment diagnosis method according to claim 1 is recorded.
